(19) United States
(12) Patent Application Publication
(10) Pub. No.: US 2022/0157402 A1
(43) Pub. Date: May 19, 2022

(54) Prediction of oscillation patterns of charges in a DNA sequence

(71) Applicants: Max Myakishev-Rempel, San Diego, CA (US); Ivan Savelev, San Diego, CA (US)

(72) Inventors: Max Myakishev-Rempel, San Diego, CA (US); Ivan Savelev, San Diego, CA (US)

(21) Appl. No.: 17/529,253

(22) Filed: Nov. 17, 2021

Related U.S. Application Data
(60) Provisional application No. 63/115,316, filed on Nov. 18, 2020.

(51) Int. Cl. G16B 40/00; G06N 10/80; G16B 50/30; C12Q 1/683; A61B 5/263

(52) U.S. Cl. CPC G16B 40/00; G06N 10/80; A61B 5/263; C12Q 1/683; G16B 50/30

(57) ABSTRACT

Some aspects of the present invention include a system for computationally prediction of oscillation patterns of charges in a DNA sequence. Such a system includes one or more means for computationally predicting proton wires with longitudinal (coaxial) hydrogen bonds in the DNA sequence; and at least one means for predicting electron wires in the DNA sequence. These wires connect the aromatic rings of DNA basepairs. The above system includes at least one means for predicting tautomeric oscillations in said DNA.

A method according to some aspects of the present invention for computationally predicting oscillation pattern of charges in a DNA sequence includes: computationally predicting proton wires containing longitudinal (coaxial) hydrogen bonds, the wires spanning at least two DNA basepairs; predicting electron wires in the DNA which includes stretches of purines; and predicting tautomeric oscillations in the DNA.

### RELATED APPLICATION/S

This application claims the benefit of priority from U.S. Provisional Patent Application No. 63/115,316 filed on Nov. 18, 2020, the contents of which are incorporated herein by reference in their entirety.

### OTHER PUBLICATIONS

Abou-Zied, O. K., R. Jimenez, and F. E. Romesberg. 2001. “Tautomerization Dynamics of a Model Base Pair in DNA.” Journal of the American Chemical Society 123 (19): 4613-14. https://doi.org/10.1021/ja003647s.
Bai, Yu, Jun Wang, Jin-Peng Wu, Jing-Xing Dai, Ou Sha, David Tai Wai Yew, Lin Yuan, and Qiu-Ni Liang. 2011. “Review of Evidence Suggesting That the Fascia Network Could Be the Anatomical Basis for Acupoints and Meridians in the Human Body.” Evidence-Based Complementary and Alternative Medicine: eCAM 2011 (April): 260510. https://doi.org/10.1155/2011/260510.
Ball, Philip. 2018. Beyond Weird: Why Everything You Thought You Knew about Quantum Physics Is Different. University of Chicago Press. https://play.google.com/store/books/details?id=Nk1vDwAAQBAJ.
Bem, Daryl, Patrizio Tressoldi, Thomas Rabeyron, and Michael Duggan. 2015. “Feeling the Future: A Meta-Analysis of 90 Experiments on the Anomalous Anticipation of Random Future Events.” F1000Research 4 (October): 1188. https://doi.org/10.12688/f1000research.7177.2.
Bohm, David. 1980. “Wholeness and the Implicate Order Routledge & Kegan Paul.” Ltd., London & Boston.
Brovarets', Ol'ha O., and Dmytro M. Hovorun. 2014. “Why the Tautomerization of the G.C Watson-Crick Base Pair via the DPT Does Not Cause Point Mutations during DNA Replication? QM and QTAIM Comprehensive Analysis.” Journal of Biomolecular Structure & Dynamics 32 (9): 1474-99. https://doi.org/10.1080/07391102.2013.822829.
Ol'ha O. Brovarets' & Dmytro M. Hovorun 2015. “Proton Tunneling in the A.T Watson-Crick DNA Base Pair: Myth or Reality?” Journal of Biomolecular Structure & Dynamics 33 (12): 2716-20. https://www.tandfonline.com/doi/abs/10.1080/07391102.2015.1092886?casa_token=Gt7MH EaQ2F0AAAAA:qbDAUdmYI036piWPKySbFdsO1B99N84QDp9KZF3_CYkroDYCvJY WzGBRzOER7AiAwh7mBn2D87Bxg.
Cifra, Michal, Jeremy Z. Fields, and Ashkan Farhadi. 2011. “Electromagnetic Cellular Interactions.” Progress in Biophysics and Molecular Biology 105 (3): 223-46. https://doi.org/10.1016/j.pbiomolbio.2010.07.003.
Corballis, Michael C. 2014. “Left Brain, Right Brain: Facts and Fantasies.” PLoS Biology 12 (1): e1001767. https://doi.org/10.1371/journal.pbio.1001767.
Deaton, Aimee M., and Adrian Bird. 2011. “CpG Islands and the Regulation of Transcription.” Genes & Development 25 (10): 1010-22. https://doi.org/10.1101/gad.2037511.
Dexter, Joseph P., Sudhakaran Prabakaran, and Jeremy Gunawardena. 2019. “A Complex Hierarchy of Avoidance Behaviors in a Single-Cell Eukaryote.” Current Biology: CB 29 (24): 4323-29.e2. https://doi.org/10.1016/j.cub.2019.10.059.
Dussutour, Audrey, Tanya Latty, Madeleine Beekman, and Stephen J. Simpson. 2010. “Amoeboid Organism Solves Complex Nutritional Challenges.” Proceedings of the National Academy of Sciences of the United States of America 107 (10): 4607-11. https://doi.org/10.1073/pnas.0912198107.
Frohlich, H. 1988. “Theoretical Physics and Biology.” Biological Coherence and Response to External Stimuli. Berlin: Springer-Verlag, 1-24. http://link.springer.com/content/pdf/10.1007/978-3-642-73309-3.pdf#page=9.
Gariaev, Peter, Boris I. Birshtein, Alexander M. Iarochenko, Peter J. Marcer, George G. Tertishny, Katherine A. Leonova, and Uwe Kaempf. 2001. “The DNA-Wave Biocomputer.” International Journal of Computing Anticipatory Systems. Ed. Daniel Dubois, Published by CHAOS 10. http://www.curealternative.net/bioelettr/memoria_acqua_DNA_wave_computer.pdf.
Guerrini, R., F. Moro, M. Kato, A. J. Barkovich, T. Shiihara, M. A. McShane, J. Hurst, et al. 2007. “Expansion of the First PolyA Tract of ARX Causes Infantile Spasms and Status Dystonicus.” Neurology 69 (5): 427-33. https://doi.org/10.1212/01.wn1.0000266594.16202.c1.
Gurwitsch, A. A. 1988. “A Historical Review of the Problem of Mitogenetic Radiation.” Experientia 44 (7): 545-50. https://doi.org/10.1007/BF01953301.
Gurwitsch, Alexander. 1922. “Über Den Begriff Des Embryonalen Feldes.” Wilhelm Roux' Archiv Fur Entwicklungsmechanik Der Organismen 51 (1): 383-415. https://doi.org/10.1007/BF02554452.
Hameroff, S. 2003. “Time, Consciousness and Quantum Events in Fundamental Spacetime Geometry.” In The Nature of Time: Geometry, Physics and Perception, edited by Rosolino Buccheri, Metod Saniga, and William Mark Stuckey, 77-89. Dordrecht: Springer Netherlands. https://doi.org/10.1007/978-94-010-0155-7_9.
Hameroff, Stuart R. 1997. “Models of Classical and Quantum Computation in Microtubules: Implications for Consciousness.” In BCEC, 193-217. https://books.google.com/books?hl=en&lr=&id=TR7QDgAAQBAJ&oi=fnd&pg=PA193&dq=Orchestrated+reduction+of+quantum+coherence+in+brain+microtubules+A+model+for+consciousnessS+Hameroff+R+Penrose+Mathematics+and+computers+in+simulation+40+(3-4)+453-480&ots=vsdTvgZNoW&sig=iueSxW9yWgoH0AwnctrWRMxW168.
Hameroff, Stuart R., Travis J. A. Craddock, and Jack A. Tuszynski. 2014. “Quantum Effects in the Understanding of Consciousness.” Journal of Integrative Neuroscience 13 (02): 229-52. https://www.worldscientific.com/doi/abs/10.1142/S0219635214400093.
Hameroff, Stuart Roy. 1974. “Ch'i: A Neural Hologram? Microtubules, Bioholography, and Acupuncture.” The American Journal of Chinese Medicine 02 (02): 163-70. https://doi.org/10.1142/50192415X74000213.
Hélène, C., J. L. Dimicoli, and F. Brun. 1971. “Binding of Tryptamine and 5-Hydroxytryptamine (serotonin) to Nucleic Acids. Fluorescence and Proton Magnetic Resonance Studies.” Biochemistry 10 (20): 3802-9. https://doi.org/10.1021/bi00796a025.
Johnson, Irudayam Maria, Halan Prakash, Jeyaguru Prathiba, Raghavachary Raghunathan, and Raghunathan Malathi. 2012. “Spectral Analysis of Naturally Occurring Methylxanthines (theophylline, Theobromine and Caffeine) Binding with DNA.” PloS One 7 (12): e50019. https://doi.org/10.1371/journal.pone.0050019.
Joshi, Sandeep. 2011. “Memory Transference in Organ Transplant Recipients.” Journal of New Approaches to Medicine and Health 19 (1). https://www.namahjournal.com/doc/Actual/Memory-transference-in-organ-transplant-recipients-vol-19-iss-1.html.
Kabelác̆, Martin, and Pavel Hobza. 2007. “Hydration and Stability of Nucleic Acid Bases and Base Pairs.” Physical Chemistry Chemical Physics: PCCP 9 (8): 903-17. https://doi.org/10.1039/B614420A.
Kratochvílová, Irena, Martin Vala, Martin Weiter, Miroslava S̆pérová, Bohdan Schneider, Ondr̆ej Páv, Jakub S̆ebera, Ivan Rosenberg, and Vladimír Sychrovský. 2013. “Charge Transfer through DNA/DNA Duplexes and DNA/RNA Hybrids: Complex Theoretical and Experimental Studies.” Biophysical Chemistry 180-181 (October): 127-34. https://doi.org/10.1016/j.bpc.2013.07.009.
Lafayette, Elizabeth Almeida, Sinara Mönica Vitalino de Almeida, Renata Virginia Cavalcanti Santos, Jamerson Ferreira de Oliveira, Cezar Augusto da Cruz Amorim, Rosali Maria Ferreira da Silva, Maira Galdino da Rocha Pitta, et al. 2017. “Synthesis of Novel Indole Derivatives as Promising DNA-Binding Agents and Evaluation of Antitumor and Antitopoisomerase I Activities.” European Journal of Medicinal Chemistry 136 (August): 511-22. https://doi.org/10.1016/j.ejmech.2017.05.012.
Liester, Mitchell B. 2020. “Personality Changes Following Heart Transplantation: The Role of Cellular Memory.” Medical Hypotheses 135 (February): 109468. https://doi.org/10.1016/j.mehy.2019.109468.
Li, J. F., and C. Dong. 2009. “Study on the Interaction of Morphine Chloride with Deoxyribonucleic Acid by Fluorescence Method.” Spectrochimica Acta. Part A, Molecular and Biomolecular Spectroscopy 71 (5): 1938-43. https://doi.org/10.1016/j.saa.2008.07.033.
Lund, E. J. 1917. “Reversibility of Morphogenetic Processes in Bursaria.” The Journal of Experimental Zoology 24 (1): 1-33. https://doi.org/10.1002/jez.1400240102.
Maegawa, Shingo. 2017. “Molecular Characteristics of Neuron-like Functions in Single-Cell Organisms.” In Brain Evolution by Design: From Neural Origin to Cognitive Architecture, edited by Shuichi Shigeno, Yasunori Murakami, and Tadashi Nomura, 25-44. Tokyo: Springer Japan. https://doi.org/10.1007/978-4-431-56469-0_2.
Marton, Soledad, Bruno González, Sebastián Rodríguez-Bottero, Ernesto Miguel, Laura Martínez-Palma, Mariana Pazos, José Pedro Prieto, et al. 2019. “Ibogaine Administration Modifies GDNF and BDNF Expression in Brain Regions Involved in Mesocorticolimbic and Nigral Dopaminergic Circuits.” Frontiers in Pharmacology 10 (March): 193. https://doi.org/10.3389/fphar.2019.00193.
Mathews, Albert P. 1903. “Electrical Polarity in the Hydroids.” American Journal of Physiology-Legacy Content 8 (4): 294-99. https://journals.physiology.org/doi/pdf/10.1152/ajplegacy.1903.8.4.294.
Maurer, Norbert, Helmut Nissel, Monika Egerbacher, Erich Gornik, Patrick Schuller, and Hannes Traxler. 2019. “Anatomical Evidence of Acupuncture Meridians in the Human Extracellular Matrix: Results from a Macroscopic and Microscopic Interdisciplinary Multicentre Study on Human Corpses.” Evidence-Based Complementary and Alternative Medicine. https://doi.org/10.1155/2019/6976892.
Meijer, Dirk K. F., and Hans J. H. Geesink. 2016. “Phonon Guided Biology: Architecture of Life and Conscious Perception Are Mediated by Toroidal Coupling of Phonon, Photon and Electron Information Fluxes at Discrete Eigenfrequencies.” NeuroQuantology: An Interdisciplinary Journal of Neuroscience and Quantum Physics 14 (4). https://www.academia.edu/download/52162519/Phonon_guided_biology_NQ.pdf.
Miller, Richard Alan, Burt Webb, and Darden Dickson. 1975. “A Holographic Concept of Reality.” Psychoenergetic Systems 1: 55-62. http://www.nwbotanicals.org/events/real.pdf.
Miller, Richard A., and Burt Webb. 1973. “Embryonic Holography: An Application of the Holographic Concept of Reality.” DNA Decipher Journal 2 (2). http://www.dnadecipher.com/index.php/ddj/article/view/26.
Morgan, T. H., and Abigail C. Dimon. 1904. “An Examination of the Problems of Physiological ‘polarity’ and of Electrical Polarity in the Earthworm.” Journal of Experimental Zoology. https://doi.org/10.1002/jez.1400010206.
Mossbridge, Julia A., and Dean Radin. 2018. “Precognition as a Form of Prospection: A Review of the Evidence.” Psychology of Consciousness: Theory, Research, and Practice 5 (1): 78. https://psycnet.apa.org/journals/cns/5/1/78/.
Nafisi, Shohreh, Firouzeh Manouchehri, Heidar-Ali Tajmir-Riahi, and Maryam Varavipour. 2008. “Structural Features of DNA Interaction with Caffeine and Theophylline.” Journal of Molecular Structure 875 (1): 392-99. https://doi.org/10.1016/j.molstruc.2007.05.010. Ol'ha, O. Brovarets', and Dmytro M. Hovorun. 2018. “Renaissance of the Tautomeric Hypothesis of the Spontaneous Point Mutations in DNA: New Ideas and Computational Approaches.” Mitochondrial DNA: New Insights, 31. https://books.google.com/books?h1=en&lr=&id=Ui-RDwAAQBAJ&oi=fnd&pg=PA31&dq=Renaissance+of+the+Tautomeric+Hypothesis+of+the+Spontaneous+Point+Mutations+in+DNA+New+Ideas+and+Computational+Approaches+Ol+ha+O+Brovarets+and+Dmytro+M+Hovorun&ots=1OVPDNNqDF&sig=eFIC-GWVP5xhbezRKKCSRX47RPw.
Pearsall, Paul, Gary E. R. Schwartz, and Linda G. S. Russek. 2002. “Changes in Heart Transplant Recipients That Parallel the Personalities of Their Donors.” Journal of Near-Death Studies 20 (3): 191-206. https://idp.springer.com/authorize/casa?redirect_uri=https://link.springer.com/article/10.1023/A:1013009425905&casa_token=uGxsEA-qtPcAAAAA:ENjBkxx_8n2RNKu9Kp6IH1h_0jGgELODSNQDCH0AiXT1dpvra1bT3qfXn PBR6hMmJItyTq3JQmrTfcZBNg.
Penrose, Roger. 1994. Shadows of the Mind: A Search for the Missing Science of Consciousness. Oxford University Press. https://play.google.com/store/books/details?id=gDbOAK89tmcC.
Pérez, Alejandro, Mark E. Tuckerman, Harold P. Hjalmarson, and O. Anatole Von Lilienfeld. 2010. “Enol Tautomers of Watson-Crick Base Pair Models Are Metastable because of Nuclear Quantum Effects.” Journal of the American Chemical Society 132 (33): 11510-15. https://pubs.acs.org/doi/abs/10.1021/ja102004b.
Pitkänen, Matti. 2010. “DNA as Topological Quantum Computer.” In TGD as a Generalized Number Theory. Matti Pitkanen, tgd.wippiespace.com. http://scireprints.lu.lv/66/.
Polesskaya, Oksana, Vadim Guschin, Nikolai Kondratev, Irina Garanina, Olga Nazarenko, Nelli Zyryanova, Alexey Tovmash, et al. 2018. “On Possible Role of DNA Electrodynamics in Chromatin Regulation.” Progress in Biophysics and Molecular Biology 134 (May): 50-54. https://doi.org/10.1016/j.pbiomolbio.2017.12.006.
Radin, Dean. 2009. Entangled Minds: Extrasensory Experiences in a Quantum Reality. Simon and Schuster. https://play.google.com/store/books/details?id=sUM1Hc-KwJQC.
Rescifina, Antonio, Chiara Zagni, Maria Giulia Varrica, Venerando Pistarà, and Antonino Corsaro. 2014. “Recent Advances in Small Organic Molecules as DNA Intercalating Agents: Synthesis, Activity, and Modeling.” European Journal of Medicinal Chemistry 74 (March): 95-115. https://doi.org/10.1016/j.ejmech.2013.11.029.
Savelev, Ivan, and Max Myakishev-Rempel. 2020. “Evidence for DNA Resonance Signaling via Longitudinal Hydrogen Bonds.” Progress in Biophysics and Molecular Biology, July. https://doi.org/10.1016/j.pbiomolbio.2020.07.005.
Savelyev, Ivan, and Max Myakishev-Rempel. 2019. “Possible Traces of Resonance Signaling in the Genome,” August. https://www.researchgate.net/profile/Max_Myakishev-Rempel/publication/335149527_Possible_traces_of_resonance_signaling_in_the_genome/links/5d5310c4299bf16f073695fd/Possible-traces-of-resonance-signaling-in-the-genome.pdf.
Savelyev, Ivan V., Nelli V. Zyryanova, Oksana O. Polesskaya, and Max Myakishev-Rempel. 2019. “On The Existence of The DNA Resonance Code and Its Possible Mechanistic Connection to The Neural Code.” NeuroQuantology: An Interdisciplinary Journal of Neuroscience and Quantum Physics 17 (2).
Scholkmann, Felix, Daniel Fels, and Michal Cifra. 2013. “Non-Chemical and Non-Contact Cell-to-Cell Communication: A Short Review.” American Journal of Translational Research 5 (6): 586-93. https://www.ncbi.nlm.nih.gov/pubmed/24093056.
Scott, A. C. 1985. “Soliton Oscillations in DNA.” Physical Review A: General Physics 31 (5): 3518-19. https://doi.org/10.1103/PhysRevA.31.3518.
Sheldrake, Rupert. 2009. Morphic Resonance: The Nature of Formative Causation. Inner Traditions/Bear & Co. https://play.google.com/store/books/details?id=F75gxeTBgocC.
Shimony, Abner. 1997. On Mentality, Quantum Mechanics and the Actualization of Potentialities. na.
Shimony, Abner, and James T. Cushing. 1994. “Search for a Naturalistic World View. Vol. 1: Scientific Method and Epistemology. Vol. 2: Natural Science and Metaphysics.” American Journal of Physics. https://doi.org/10.1119/1.17617.
Smythies, J. R., F. Benington, and R. D. Morin. 1970. “The Mechanism of Action of Hallucinogenic Drugs on a Possible Serotonin Receptor in the Brain.” International Review of Neurobiology 12: 207-33. https://doi.org/10.1016/s0074-7742(08)60062-8.
Storm, Lance, Simon J. Sherwood, Chris A. Roe, Patrizio E. Tressoldi, Adam J. Rock, and Lorenzo Di Risio. 2017. “On the Correspondence between Dream Content and Target Material under Laboratory Conditions: A Meta-Analysis of Dream-ESP Studies, 1966-2016.” International Journal of Dream Research 10 (2): 120-40. https://psycnet.apa.org/fulltext/2018-14483-003.pdf.
Talemi, Rasoul Pourtaghavi, and Mohammad Hossein Mashhadizadeh. 2015. “A Novel Morphine Electrochemical Biosensor Based on Intercalative and Electrostatic Interaction of Morphine with Double Strand DNA Immobilized onto a Modified Au Electrode.” Talanta 131 (January): 460-66. https://doi.org/10.1016/j.talanta.2014.08.009.
Tian, Zhengshan, Zechun Wang, Xinxin Han, Ning Wang, and Ruiyong Wang. 2018. “Study on the Interaction between Cannabinol and DNA Using Acridine Orange as a Fluorescence Probe.” Journal of Molecular Recognition: JMR 31 (2). https://doi.org/10.1002/jmr.2682.
Trushin, Maxim V. 2004. “Distant Non-Chemical Communication in Various Biological Systems.” Rivista Di Biologia 97 (3): 409-42. https://www.ncbi.nlm.nih.gov/pubmed/15754593.
Vignoni, Mariana, Rosa Erra-Balsells, Bernd Epe, and Franco M. Cabrerizo. 2014. “Intra- and Extra-Cellular DNA Damage by Harmine and 9-Methyl-Harmine.” Journal of Photochemistry and Photobiology. B, Biology 132 (March): 66-71. https://doi.org/10.1016/j.jphotobiol.2014.01.020.
Volkov, S. N., and A. M. Kosevich. 1987. “Conformation Oscillations of DNA.” Molekuliarnaia Biologiia 21 (3): 797-806. https://europepmc.org/abstract/med/3657778.
Volodyaev, Ilya, and Lev V. Beloussov. 2015. “Revisiting the Mitogenetic Effect of Ultra-Weak Photon Emission.” Frontiers in Physiology 6 (September): 241. https://doi.org/10.3389/fphys.2015.00241.
Wink, M., T. Schmeller, and B. Latz-Brüning. 1998. “Modes of Action of Allelochemical Alkaloids: Interaction with Neuroreceptors, DNA, and Other Molecular Targets.” Journal of Chemical Ecology 24 (11): 1881-1937. https://idp.springer.com/authorize/casa?redirect_uri=https://link.springer.com/article/10.1023/A:1022315802264&casa_token=hcRaCBPWqJgAAAAA:en7s9Wb0_tvtPxjCJLoDqTfpMS 06NPph2E_eWpqh5L2RQ8FE9VjuWSbNJDW0eV1sjLyk17VeddgKDmvnNA.
Xu, Jingjing, Fan Yang, Danhong Han, and Shengyong Xu. 2017. “Wireless Communication in Biosystems.” arXiv [physics.bio-Ph]. arXiv. http://arxiv.org/abs/1708.02467.
Zheng, Sai-Jing, and Xiang-Qin Lin. 2003. “Spectral and Electrochemical Investigation of Intercalations of Adrenaline and CT-DNA.” Chinese Journal of Chemistry 21 (7): 767-71. https://doi.org/10.1002/cjoc.20030210712.

### TECHNICAL FIELD

The present invention relates to computational genomics, more particularly, to the prediction of oscillation patterns of charges in DNA sequence.

### BACKGROUND

The role of DNA in the work of the mind is currently mostly thought limited to protein-coding genes, which are dynamically regulated and which, in turn, regulate the levels of proteins which in turn regulate the work of the brain. This mechanism is indirect, slow, and seems insufficient to explain the complexity and speed of our thinking Previously, the inventors of the present invention suggested that there is a much faster and more direct mechanism by which DNA is involved in the thinking process (I. V. Savelyev et al. 2019).

The exclusivity of the neuronal signaling mechanism for thinking is challenged by simple organisms that don't have neurons or have only a few neurons. The nematode Caenorhabditis elegans has only 302 neurons but displays several complex behaviors including predator escape and mating. Some single-cell organisms having no neurons also demonstrate the ability of learning and complex behaviors. Moreover, free-living single-cell ciliates such as Stentor roeselii are capable of learning (Dexter, Prabakaran, and Gunawardena 2019) as well as Plasmodium, which is a single large cell with many nuclei (Dussutour et al. 2010). Paramecium, a single cell organism can swim, learn, display complex behaviors, and sexually reproduce (Maegawa 2017). This demonstrates that there are subcellular structures capable of thinking and making decisions (Maegawa 2017).

The role of electric fields in morphogenesis was developed by Mathews (Mathews 1903), Morgan (Morgan and Dimon 1904), Lund (Lund 1917), (A. Gurwitsch 1922), and others over 100 years ago. It was proposed that the morphogenetic field is produced by the union of the cells of the organism, this field guides the development of the shape of the body and regulates the function of each part and organ by instructions delivered to organism cells. The existence of the morphogenetic field was experimentally demonstrated by independent groups (A. A. Gurwitsch 1988; Volodyaev and Beloussov 2015). In these experiments, perturbing one of the chemically separated biological samples lead to measurable effects in another (Cifra, Fields, and Farhadi 2011; Scholkmann, Fels, and Cifra 2013; Trushin 2004; Xu et al. 2017). The electromagnetic oscillations in the cells were proposed to be driven by the constant chemical energy flux and were estimated to be in the millimeter-wave region (Frohlich 1988). Miller and Web proposed that genomic DNA is the main source and receiver of the morphogenetic field, allowing the genomic program to direct the morphogenesis directly via a holographic electromagnetic field (Miller et al., 1975; Miller and Webb, 1973). Moreover, it was proposed that through the same field, the genomic DNA of brain cells is directly involved in the work of the mind (Richard Alan Miller, Webb, and Dickson 1975). Hameroff proposed that microtubules in axons work as light guides and are transmitting information in neurons thus explaining the high speed and bandwidth of the mind (Stuart Roy Hameroff 1974). The inventors of the present invention had combined and expanded the ideas of Miller, Webb, and Hameroff by suggesting an electromagnetic information transfer between the DNA in the nucleus, microtubules in the cytoplasm, and the fibers of the extracellular matrix in the fascia (I. V. Savelyev et al. 2019).

Although scarcely known in the west, there is a substantial body of experimental evidence that there exists a system of signaling that exchanges electromagnetic signals via fiberoptic-like tubular structures of fascia tissue that wraps and penetrates the whole body and thus, is perfectly placed to regulate the body's growth and health (Maurer et al. 2019; Bai et al. 2011). The inventors of the present invention proposed that the genome copies of each cell of the body are vibrationally coupled with the signaling system of meridians in the fascia and thus are linked into one fiberoptic network (I. V. Savelyev et al. 2019). The frequencies of the waves in this network may be in the infrared and millimeter-wave regions (I. V. Savelyev et al. 2019).

For the genome copies to communicate via electromagnetic waves, DNA fragments should be able to resonate in a sequence-dependent manner Although mechanical oscillations in DNA have been proposed (Scott 1985; Volkov and Kosevich 1987), The inventors of the present invention reasoned that the mechanical oscillations would be damped by the viscosity of the nucleoplasm. Instead, the inventors of the present invention proposed that there must be oscillations of delocalized charges in the nucleobase stack which would be protected from dumping by the DNA backbone. Specifically, the inventors of the present invention predicted the existence of proton and electron clouds in the base stack. Then the inventors of the present invention modeled their approximate shapes and published the evidence for evolutionary selection and conservation of certain sequences that code for specific shapes of electron and proton clouds in the base stack (I. Savelyev and Myakishev-Rempel 2019; Savelev and Myakishev-Rempel 2020).

### SUMMARY OF EMBODIMENTS OF THE INVENTION

Previously the authors proposed that DNA is involved in the work of mind directly and immediately via the network of optical fibers. The authors proposed the mechanism of signal transduction in DNA via a sequence-specific resonance between the clouds of delocalized charges in the base stack. It was computationally demonstrated that certain repetitive patterns of delocalized charge clouds were evolutionarily enriched in various genomes. Here, the authors propose that natural quantum computation in DNA in living cells is based on the tautomerization of basepairs and involves coordinated oscillations of hydrogen-bond protons and aromatic electrons. The authors expand the ORCH-OR theory to include the collapse of the wave function of aromatic electrons in purines and propose that such collapses and expansions produce the experience of consciousness and the perception of time. Quantum mechanical considerations for the collapse of aromaticity by double proton transfer in basepairs are discussed in terms of the collapse of the wave function, loss of delocalization, and the dynamic balance between coherence and decoherence in DNA.

The present invention encompasses prediction of psychoactive activity of a drug based on its aromatic structure and the way it modifies the aromaticity of DNA. This prediction can be made based on computational modeling or spectroscopic measurement of how the drug modifies the aromaticity of DNA.

It is within the scope of the present invention, artificial quantum computers based on the aromaticity collapse in DNA.

Some embodiments of the present invention include phase-shifted flashed of lights into the eyes, and pulses of sound into the ears to treat psychological problems and improve mental performance utilizing DNA biofield which is involved in thinking, consciousness, and psychology. Interacting with biofield may lead to improved psychological and mental performance. It may involve optimization and combination of multiple modalities.

Some embodiments of the invention include genetic analysis of genomic variations responsible for psychological traits for the prediction of psychological traits and mental performance. This analysis can include detection of DNA sequence-specific electron and proton chains.

Some aspects of the present invention include a system for computationally prediction of oscillation patterns of charges in a DNA sequence. Such a system includes one or more means for computationally predicting proton wires with longitudinal (coaxial) hydrogen bonds in the DNA sequence; and at least one means for predicting electron wires in the DNA sequence. These wires connect the aromatic rings of DNA basepairs. The above system includes at least one means for predicting tautomeric oscillations in said DNA.

According to some embodiments of the present invention, the above DNA sequence is a processor of a quantum computer. The quantum computer can be topological and spintronic, and utilize spins of purines aromatic electrons. In some cases the tautomerization of the basepairs is utilized for performing logical operations. The aforementioned system may include logical circuits created by the DNA longitudinal hydrogen bonds. The hydrogen bonds are connected to basepairs with proton wires and electron wires. The DNA sequence may include at least one purine stretch patterned in periodic tandem arrays. The pattern may enable coordinated oscillations of aromaticity in the purine stretches.

In some examples embodying the present invention information input is received in the DNA processor which converts the information input into oscillations such as electromagnetic, electroacoustic, and combinations thereof. Output from the logical operations is obtained by at least one electronic module adapted to detect at least one change in conformation, topology and supercoiling of the DNA.

The above mentioned processor may be incorporated into at least one living cell. At least one probe may be connected to a subject body. Such probe may include at least one DNA sequence matching resonance frequencies caused by at least one subject disorder; at least one sensor adapted to measure at least one change in the DNA e.g., conformational, topological, supercoiling; and at least one processor adapted to receive and analyze input from the sensor that is related to the measurement of a change in the DNA. In such case it is desirable that the DNA sequence matches resonance frequencies caused by at least one subject disorder.

A method according to some aspects of the present invention for computationally predicting oscillation pattern of charges in a DNA sequence includes: computationally predicting proton wires containing longitudinal (coaxial) hydrogen bonds, the wires spanning at least two DNA basepairs; predicting electron wires in the DNA which includes stretches of purines; and predicting tautomeric oscillations in the DNA.

The method may include incorporating the DNA sequence as a processor in a quantum computer and utilizing spins of purines aromatic electrons for computing. In some cases the method includes incorporating said processor in at least one living cell. In some examples the method includes utilizing tautomerization of the basepairs for performing logical operations and utilizing periodic tandem arrays of the purines to improve efficiency of electromagnetic oscillations in the electron wires.

In some examples embodying the invention the method includes utilizing waves produced by the DNA sequence for therapy. These waves may be utilized for improving mental performance. The waves may be utilized for communicating with at least one brain.

The method may include modifying the above mentioned patterns according to electric and bioimpedance measurements made in parts of a subject body.

### BRIEF DESCRIPTION OF DRAWINGS

Preferred embodiments, features, aspects and advantages of the present invention are described herein in conjunction with the following drawings:

FIG. 1. schematically depicts tautomeric forms of GC basepair

FIG. 2. schematically depicts tautomeric forms of AT basepair

FIG. 3. schematically depicts aromatic electron rings are merged in a purine stretch

FIG. 4. schematically depicts electron and proton jumps

FIG. 5. schematically depicts Proton wires span multiple bases

FIG. 6. is a list of main psychoactive substances according to the types of aromatic groups they contain, their aromaticity, their similarity to nucleobases. Also included are two intercalating substances: ethidium and SYBR Green for which psychoactive effects are unknown.

FIG. 7. schematically depicts stability and aromaticity oscillations of purine tautomers in basepairs

### DETAILED DESCRIPTION OF EMBODIMENTS

The inventors of the present invention believe that the old picture of slow and indirect involvement of DNA in the work of the mind should be supplemented by a model of the direct and fast signaling between DNA of nuclei of the body via electromagnetic waves. It involves charge oscillations in DNA and the exchange of electromagnetic signals between cell nuclei via a network of microtubules and other fibers.

The inventors of the present invention have been addressing the dissipation and scattering of electromagnetic signals in the tissues. Thus, two models are considered for the exchange of electromagnetic signals between the DNA of cell nuclei: (1) the field model in which the genomes radiate an electromagnetic field in all directions and (2) the fiberoptic model (I. V. Savelyev et al. 2019) where the genomes exchange the electromagnetic signals via the network of microtubules and other fibers. So far, the experimental evidence is published only for the field model (Cifra, Fields, and Farhadi 2011; Scholkmann, Fels, and Cifra 2013; Trushin 2004; Xu et al. 2017). In these publications, the field and information transfer was demonstrated, yet the role of DNA in its generation and reception was not tested. The inventors of the present invention had envisioned that both field and fiberoptic models coexist side by side, some signals are exchanged via microtubules and some signals are exchanged via the field. The fiberoptic model has the advantage that it minimizes data loss and crosstalk: information can be exchanged between specific locations without loss, contamination, and crosstalk. Importantly, the fiberoptic model perfectly corresponds to the meridians in Traditional Chinese Medicine.

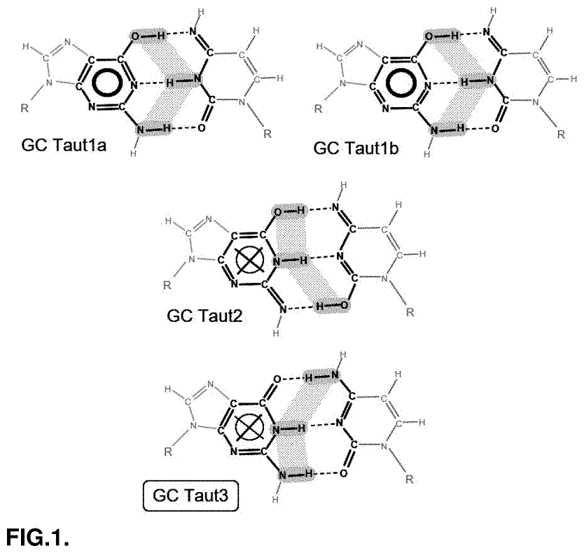

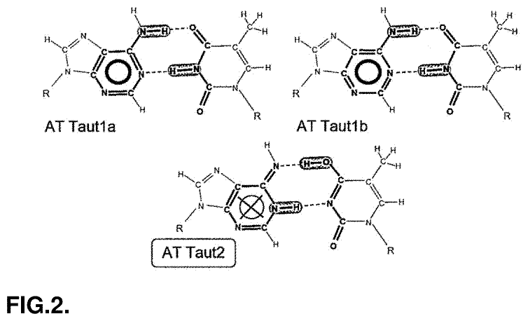

The inventors of the present invention had contemplated that key resonators, transmitting, and receiving antennas are repetitive elements in DNA that comprise over 50% of our genome. The vibrational information is coded in positions of repetitive elements, variations within them, and in their flanking sequences. The repetitive elements work as radios by converting biomolecular information into electromagnetic wave messages and back. They create an interference pattern of waves that is united between all cells of the organism, guides its development, and is an integral part of the work of the mind. The wave signals that are received by the DNA resonance elements are guiding the expression of genes and chromatin dynamics. Some embodiments of the present invention include utilization of DNA in the co-creation of consciousness. Some embodiments of the invention involve utilization of hydrogen bonds and electrons in tautomeric forms of the DNA basepairs. Some embodiments of the invention involve utilization of delocalized charged particles in DNA basepairs. The shapes of delocalized charge may depend on the DNA sequence. The sequence dependence of the delocalized charge clouds may function as oscillators that may mediate electromagnetic signaling in the body. The normal coexistence and interconversion of tautomeric forms of normal Watson-Crick basepairs are known from experiments in model systems (Abou-Zied, Jimenez, and Romesberg 2001), illustrated in FIG. 1 where tautomeric forms of GC basepair are represented. The hexagonal heterocycles of purines are called “the central ring” and labeled with circles. The uncrossed circles signify aromaticity and the crossed-out circles signify the loss of aromaticity. The remaining heterocycles are not aromatic. The thick lines signify the structures that undergo tautomerization and thin lines signify the structures that stay unchanged during tautomerization. Links to the backbone are shown as “R”. From the classical chemistry perspective, the tautomers naturally transform into each other with the frequencies of 10 GHz and higher. From the quantum chemistry perspective, the tautomeric forms coexist in the state of quantum superposition until they are measured or “chemically forced to make a choice” in which state they exist. Both classical chemistry and quantum chemistry perspectives are true at the same time. As illustrated in FIG. 1, it takes two proton relocations to switch the basepair from Taut1 to Taut2, from Taut2 to Taut3, and from Taut3 to Taut1. It can be seen that each switch between forms Taut1, Taut2, and Taut3 is also accompanied by electron relocations. As one positively charged proton jumps one step, two or more electrons in a chain jump one step each towards this proton to rebalance the electrical charge and keep the charge of each of the bigger atoms neutral. Although the two 6-atom and one 5 atom-rings in the basepair may look similar and have alternating double bonds, only one ring is aromatic, the 6-atom ring of the purines A or G, further called “the central ring”. Its aromaticity is classical and characterized by the unification of 6 pi electrons of the ring into one delocalized cloud. The other two rings don't have enough pi-electrons to create a delocalized ring and therefore are not aromatic. The central ring is only aromatic in Taut1 and is not aromatic in Taut2 and Taut3. This happens because the relocation of protons causes the relocation of electrons and the ring loses an electron in the middle to a proton that attaches to the purine in the middle. In Taut1, the electrons of the central ring exist in a superposition of two configurations Taut1A and Taut1B. A similar dependence of the aromaticity of the central ring on the relocations of protons is observed in the classical Watson-Crick's basepair AT, illustrated in FIG. 2 with representative shapes similar to FIG. 1. The main difference is that in AT, there are only two forms Taut1 and Taut2. The form Taut1 enables aromaticity and the form Taut2 disables it. The inventors of the present invention had realized that the central ring gains and loses the aromaticity from the jumping of protons and had realized that this phenomenon is related to the role of DNA in the work of the mind. In the aromatic state the 6 pi electrons are delocalized forming a stable ring that freely spins. The spinning of the ring creates a magnetic moment and vice versa, applying a magnetic field to the electron ring spins it (The induced magnetic field in cyclic molecules. Merino, G.; Heine, T.; Seifert, G. Chem. Eur. J.; 2004; 10; 4367-4371. doi:10.1002/chem.200400457). The stacked aromatic rings are attracted via a known effect of aromatic ring stacking and their magnetic moments unite thus stabilizing the system. The switches can happen by reversing the rotation and correspondingly the polarity of the rings. The attraction of rings spinning in the same direction and magnetized in the same direction could straighten the DNA, while repulsion of the rings spinning in the opposite directions and magnetized in the opposite directions could bend or expand the DNA. Therefore applying ultrahigh-frequency alternating voltages to the DNA could control its supercoiling. Loops containing multiple genes are uncoiled by gyrases, the genes are transcribed and then the loops are coiled back. Similarly, the coiling of DNA happens during replication. Thus regulation of coiling is an essential process in gene and chromatin regulation. The inventors of the present invention suggest that in addition to gyrases, the cell uses alternating voltages of high frequency to control the coiling and uncoiling of DNA, or in other words, the DNA controls its own coiling.

The proton jump, locking and unlocking the spinning electron rings of the central ring, may function as a natural mechanism for the ability of DNA “to think”. The proton jumps cause the collapse and expansion of the wave function of the aromatic electrons of the central ring. Or in other words, the proton jumps force these aromatic electrons to localize and delocalize. Such “collapses of the wave function” were proposed to be the mechanism underlying the work of mind and consciousness (Shimony and Cushing 1994; Shimony 1997). On the molecular level, this idea was developed by Hameroff (S. Hameroff 2003) for microtubules. Here, the inventors of the present invention propose that it is DNA and more specifically, the localization and delocalization of aromatic electrons in the central rings of purines is a mechanism for our thinking process.

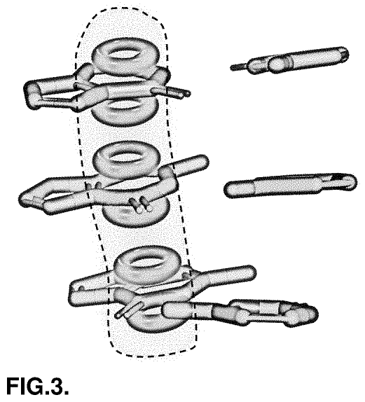

Terrence McKenna suggested that it was the evolution of hominids from gathering to hunting that forced us to develop our logical mind. He suggested that it is our predatory nature that requires us to logically create plans and execute them, otherwise, we wouldn't survive. This makes modern civilization prefer logic over intuition and action over passivity. McKenna noted that primitive tribes have a different mindset which is much more passive and intuitive. Ego, logic, and choice-making are considered in popular culture as left-brain, these would correspond to the localized state of aromatic electrons and the loss of aromaticity in a model that had been contemplated by the inventors of the present invention. Similarly, selflessness, intuition, and passivity are in popular culture attributed to right-brain, although the extent of brain asymmetry is exaggerated in the popular culture (Corballis 2014), these would correspond to the delocalized state and aromaticity of central ring electrons in our model. Moreover, when in the DNA sequence multiple purines follow each other, FIG. 3, their aromatic rings are attracted by the stacking forces and their magnetic moments would tend to unite and face in one direction. This would likely create a delocalized cloud of aromatic electrons spanning this stretch of purines and thus create a structure prone to charge oscillations. Since stretches of purines are frequent in the genome, it is suggested here that chains of purines would create antennas that would allow for wireless communication between the parts of the genome and between the genomes of all cells in the body. Thus, the delocalized state of electrons in purine stretches would allow for organism-wide resonances and nonlocal communications which nicely match the intuitive state of mind. Conversely, the collapse of the wave function and the loss of aromaticity in purine stretches would correspond to the logical way of thinking and making choices.

One peculiarity of the delocalization of charges in the basepair shown in FIG. 1, is that not only electrons are delocalized, but also protons. Quantum delocalization of protons in basepairs is known from molecular dynamic calculations (Perez et al. 2010). Since they are 800 times heavier than electrons, their delocalization is also less pronounced, but still, is real. Both protons and electrons exist in the state of delocalization, quantum superposition, and obey Heisenberg's uncertainty principle. In the basepair's natural state and outside observer not only can not say the position of electrons of the central ring but also the position of the protons of the hydrogen bonds—the electrons are fuzed together in a double electron ring above and below the central ring and protons are delocalized into a probability cloud spreading along the hydrogen bond. From a quantum mechanical perspective, the tautomer's shown in FIG. 1 are superimposed on each other and co-exist at the same time. Yet, from a chemistry perspective, the basepair switched between 3 tautomers with a frequency of around 10 GHz. Both perspectives are true and coexist at the same time.

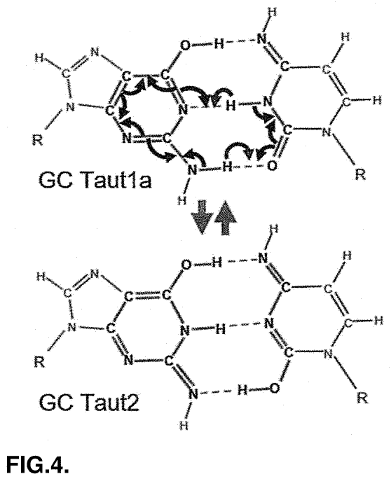

Also puzzling is the dependence of the central ring aromaticity on the tautomers. Only tautomer Taut1 enables the central ring to become aromatic, while Taut2 and Taut3 disable the aromaticity of the central ring. Yet, all three tautomers coexist at the same time and are in a state of quantum superposition. This paradox is unresolvable from the perspective of the deterministic logic of the macroworld, so it can only be accepted as a gimmick of the quantum world. To illustrate the paradox let's expand the analogy of Schrödinger's cat. The cat can exist in 3 positions Taut1, Taut2, and Taut3 corresponding to 3 tautomers of the GC basepair. For the outside observer, the position of the cat is unknown, until the observer opens the box. Now, the cat also is an observer and this represents Taut1. The cat observes a smaller closed box containing a self-spinning carousel with 6 mice representing 6 electrons of the central ring. FIG. 4. schematically depicts delocalized electrons. The mice are labeled with numbers. The spinning of the wheel represents the delocalization of the electrons. The cat once in a while opens the smaller box and grabs two mice from the carousel and the remaining 4 mice hide in 4 corners. This represents the loss of aromaticity. If the cat grabs the 2 mice with two paws, this represents his second position and Taut2. If the cat grabs the 2 mice with the right paw and the mouth, this represents his third position and Taut3. The cat reads the numbers of the mice and lets them go switching back to the first position Taut1 and the 6 mice again start spinning on the carousel. Therefore, we can see that the quantum effects (uncertainty, delocalization, and superposition) are embedded. The human observer observes a cat which observes the mice. According to this model the mice are delocalized (superimposed and uncertain) only when the cat is in the position Taut1. In the other two positions, the mice are fixed, localized, their positions are certain. Therefore the human observer observes a delocalized cat that observes the delocalization of mice only part-time.

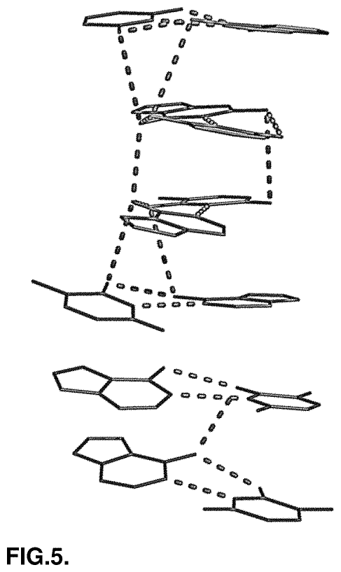

In addition to electron clouds in purine stretches, the inventors of the present invention predicted the existence of delocalized proton clouds (sometimes called proton wires) spanning multiple nucleotides in the DNA chain and obtained preliminary evidence for their existence, (Savelev and Myakishev-Rempel 2020). Accordingly proton clouds also serve as antennas for wireless communication alongside electron clouds. Hence, there exists an interplay of partially overlapping delocalized positive proton and negative electron clouds that are attracted to each other and oscillate in harmony or disharmony with each other (Polesskaya et al. 2018; I. Savelyev and Myakishev-Rempel 2019; Savelev and Myakishev-Rempel 2020). Their oscillations would only partly overlap in frequency since protons are 800 times heavier than electrons. Now, consider the interplay and coordination between these oscillations of delocalized charge clouds spanning multiple basepairs and oscillations between tautomeric forms in each basepair, illustrated in FIG. 5 with proton wires in generally vertical dashed lines. These oscillations would be linked to each other in at least two ways: delocalization of central ring electrons will provide electrons for the electron clouds and jumps of the protons within the basepair would affect strongly the structure of the proton clouds since these share the protons with the basepairs. This may suggest underlies the thinking intuitive and logical process of the DNA, of us humans, and of all life.

Let's now consider the ways how the localization and delocalization of central ring electrons can communicate with the biochemical processes outside of the base stack. Consider collective delocalization of electrons in the stretch of purines. This would make the purines aromatic and attract each other via stacking interactions. This could shrink and bend the double helix thus affecting the structure of DNA which in turn could change gene expression especially if the changes are happening in a gene promoter. Similarly, jumps of protons in basepairs could create proton clouds spanning multiple bases and this would also shrink and bend the double helix again leading to changes in chromatin structure and gene expression. Another way of affecting biochemistry is via electromagnetic oscillations. Charge oscillations that the inventors of the present invention suggest occur in electron and proton clouds spanning multiple bases can add together and their lower harmonics in the MHz-GHz range can induce ultrasound waves in the nucleoplasm. The frequency of 214 MHz corresponds to the sound wavelength of 7 um, the size of the nucleus. 750 GHz corresponds to the sound wavelength of 2 nm, the diameter of the double helix. DNA comprises a large part, about 1.5% of the nucleus mass. If a large part of the genome creates harmonized oscillations, these oscillations would create a moving sound interference pattern within the nucleus according to the theory of cymatics, reviewed in (Meijer and Geesink 2016). This way the genome could move itself using cymatic propulsion and control the movements of the proteins inside the nucleus. The reverse would be also possible—the interaction can be bidirectional—the DNA could sense the environment by interacting with the wave patterns and adjust it at will. The binding of proteins and nucleosomes to a DNA locus will radically change its vibrational properties and thus biochemical information would be converted to wave information that DNA is delivering. Conversely, the charge oscillations in DNA will modify its preference for binding nucleosomes and proteins and thus would affect the biochemical activity of a DNA locus. Moreover, charge oscillations could drive the opening and closing of chromatin thus directly controlling gene transcription.

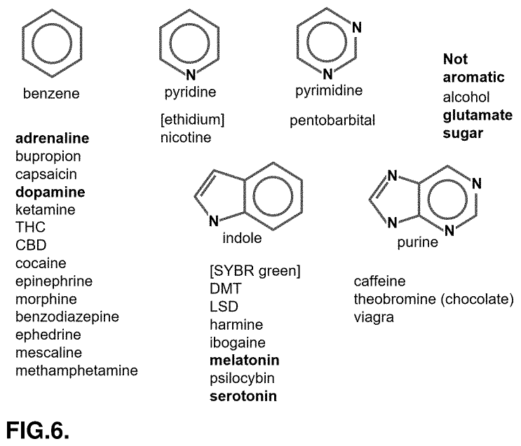

The proposed molecular mechanism for thinking and consciousness involves the oscillation of aromaticity in DNA. Classically, the psychoactive effects are being explained via binding of the drugs to proteins and blocking neurotransmitter reuptake, inhibiting neurotransmitter synthesis and inhibiting enzymes. In addition, it was proposed that psychoactive substances being predominantly aromatic, work by binding to DNA and changing its aromaticity and quantum delocalization of electrons (Smythies, Benington, and Morin 1970). Smythies pointed out that most of the psychoactive drugs contain aromatic groups similar to nucleobases, easily penetrate via cellular and nuclear membranes and can bind to DNA either via intercalation or via hydrogen bonds (Smythies, Benington, and Morin 1970). Miller highlighted the significance of electron delocalization and aromaticity in DNA for the phenomena of life” (Richard Alan Miller, Webb, and Dickson 1975). Hameroff observed a correlation between the aromaticity strength of anesthetic compounds and their potency (Stuart R. Hameroff, Craddock, and Tuszynski 2014). FIG. 6 is a list of main psychoactive substances according to the types of aromatic groups they contain, their aromaticity, their similarity to nucleobases. Also included are two intercalating substances: ethidium and SYBR Green for which psychoactive effects are unknown.

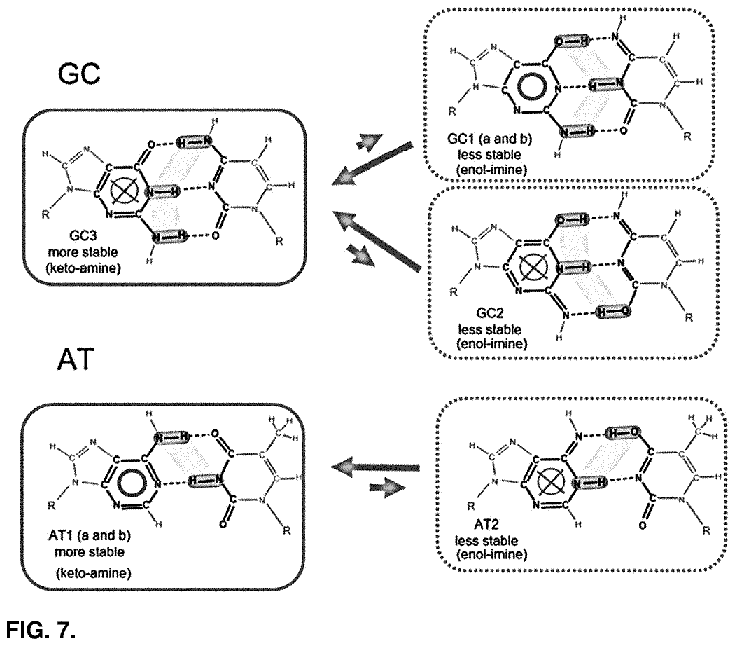

Classical Watson-Crick keto-amine tautomeric forms (GC-Taut3 and AT-Taut1, marked with continuous border on FIG. 7 with representative shapes similar to FIG. 1. These Watson-Crick keto-amine tautomeric forms are more stable than enol-imine forms (dotted border) (Pérez et al. 2010). The frequency of tautomerization was estimated using fluorescence spectroscopy in model systems (Pérez et al. 2010; Abou-Zied, Jimenez, and Romesberg 2001) and molecular dynamics calculations (Ol'ha and Hovorun 2018; Brovarets' and Hovorun 2014, 2015) and range from 104-1014 Hz, typically 0.1-10 GHz. Further understanding of tautomerization of basepairs in DNA can be done using two-dimensional Fourier-transform infrared spectroscopy Consider that tautomerization could be aperiodic or subject to complex oscillations, so the frequency estimate doesn't necessarily imply regularity in oscillations. The lifetime of more stable (keto-amine) tautomers is estimated to be about 100 times longer than of less-stable (enol-imine) tautomers, so the oscillations have a character of short pulses. The inventors of the present invention have noticed that aromaticity loss in GC and AT pairs goes in opposite directions, FIG. 7. The more-stable GC form (GC-Taut3) has a lowered aromaticity and it occasionally pulses into a less-stable GC Taut1 which is fully aromatic, that is the largely nonaromatic GC undergoes occasional short aromaticity bursts. The more-stable AT form (AT-Taut1) is fully aromatic and it occasionally pulses into a less-stable GC Taut1 with lowered aromaticity, that is the largely aromatic GC undergoes occasional short aromaticity losses.

Among functionally important and abundant genomic elements, genomic polyA tracts and CpG islands stand out. PolyA tracts are important for viruses and transposons and often a deletion of polyA tracts impairs gene function (Guerrini et al. 2007). CpG islands are typically located in genes and gene promoters and are involved in the regulation and activation of gene transcription (Deaton and Bird 2011). Based on the above observation of opposite character of aromaticity oscillations between GC and AT basepairs, it is possible to predict that polyA tracts should have a uniform stack of pi-electron rings of adenines which are 99% of the time in aromatic state and occasionally, 1% of time lose the aromaticity. Since the pi electrons in the basestack are organized in a periodic structure, they very likely exist as an organized electron cloud and their aromaticity loss might be coordinated within the polyA tract. Both high aromaticity of the uniformly periodic basestack and occasional coordinated loss of aromaticity might have an effect on their oscillatory and biomolecular function due to possible effects on DNA structure, packing of chromatin, binding of nucleosomes, and protein factors. Just the opposite should happen to CpG islands made exclusively of GC basepairs. They should exist in a reduced aromaticity state for 99% of the time and collectively burst into an aromatic state 1% of the time. This could also affect their DNA resonance signaling and also biomolecular functions.

There are several mechanisms that would predict coordination between aromaticity oscillations within stretches basepairs in DNA. First, aromatic pi-electron rings of purines unite into a periodic pattern especially when the sequence is periodic such as in tandem genomic repeats. The stacking of pi-electron rings is thought to be responsible in part for the experimentally observed high electrical conductivity of DNA in physiological conditions (Kratochvílová et al. 2013). Second, as previously published by the authors, basepairs are likely bound by delocalized proton wires made of longitudinal hydrogen bonds (Savelev and Myakishev-Rempel 2020), which could also coordinate tautomerization and aromaticity oscillations. Third, the excitations caused by tautomerization could be transmitted via the sugar-phosphate backbone and lead to coordination between basepairs. Therefore, it is likely that aromaticity oscillations are coordinated within stretches of basepairs. Since both stacking of aromatic electron rings and the formation of longitudinal hydrogen bonds depends on DNA sequence, the coordination of aromaticity oscillations would also be highly sequence-dependent. Various sequences would provide different aromaticity oscillation patterns. The aromaticity oscillation pattern of a specific DNA fragment would be defined by the interplay of aromatic pi-electron stacks and proton wires, which would be highly variable. Yet, identical sequences may have identical aromaticity oscillations patterns and synchronize with each other thus providing a mechanism for resonance signaling.

Indole derivatives such as melatonin, harmine (Vignoni et al. 2014) and ibogaine migrate into the nucleus and bind to DNA. Small aromatic molecules such as psychoactive substances listed in FIG. 6 easily penetrate the cell and nuclear membranes (Lafayette et al. 2017). Most of them bind to DNA (Rescifina et al. 2014). Other indole derivatives also bind to DNA (Lafayette et al. 2017). Hallucinogen ibogaine enters the nucleus and regulates gene expression (Marton et al. 2019). Caffeine and chocolate's theobromine bind to DNA via hydrogen bonds (Johnson et al. 2012; Nafisi et al. 2008). Cannabinol (CBN) from cannabis binds in the major groove of DNA and doesn't intercalate into it (Tian et al. 2018).

When an aromatic small molecule intercalates into DNA, it inserts itself into the base stack as if it was an additional basepair in the DNA and its aromatic ring of pi-electrons is fuzed into the periodic set of pi-electron rings of the nucleobases (Rescifina et al. 2014). Morphine binds and intercalates into DNA (Li and Dong 2009; Talemi and Mashhadizadeh 2015). Adrenaline binds to DNA and may intercalate into DNA (Zheng and Lin 2003). Hallucinogen harmine penetrates into the nucleus, binds to DNA (Vignoni et al. 2014) via intercalation (Wink, Schmeller, and Latz-Brüning 1998). Serotonin and tryptamine intercalate into DNA (Hélène, Dimicoli, and Brun 1971).

The delocalized state of aromatic electrons and protons in biological systems is described by Schrödinger's wave function. The loss of delocalization results in the collapse of Schrödinger's wave function and according to “objective reduction” (“OR”) of the quantum state this collapse is a choice and collectively these choices produce conscious awareness (Penrose 1994). This was expanded to the Orchestrated Objective Reduction (“ORCH-OR”) theory of Penrose and Hameroff (Stuart R. Hameroff 1997) which proposed the role of microtubules. There, the aromatic rings of aromatic aminoacids tyrosine, phenylalanine and tryptophan of tubulin were suggested to periodically collapse and expand producing choices and thus creating conscious awareness. Hameroff also posted online an unfinished paper suggesting the role of DNA in the process.

Here, the inventors of the present invention expand the ORCH-OR theory to include DNA. DNA and microtubules share aromatic and helical nature and their dimensions are comparable. DNA is plausible as a thinking machine since it carries the genetic code and has an efficient addressing system—it is often sufficient to know only 15 bases of the code to find a specific spot in the 3.2 billion bases of the genome. ORCH-OR theory proposes that the periodic collapse of the wave function of the aromatic aminoacids results in thinking and consciousness. Here, it is proposed the same for the aromatic electrons of the purines in DNA. In this process, periodically the aromatic tautomers Taut1 transform to nonaromatic tautomers Taut2 and Taut3, FIG. 1 and back, the electrons become localized and delocalized, their wave function collapses and expands. This can take place in each of the 6.4 billion purines in the cell. This number can be multiplied by 80 billion neurons in the brain or up to 30 trillion cells of our body, considering that not only brain neurons are involved in the thinking process.

As was proposed previously by the authors (I. V. Savelyev et al. 2019), the genomes of the body located in the nuclei are informationally coupled to the microtubules located in the cytoplasm and between the cells and thus all DNA and microtubules of the body are united into one conscious network. Hameroff also proposed that occasional wave function collapses produce time as a byproduct of creating consciousness (S. Hameroff 2003). It is suggested herein that it is the experience of time and self-awareness that is produced by the wave function collapses. Non-biological objects and unidirectional processes also exist in the space-time of our universe, but it is suggested now that it is the wave function collapses and expansions of aromatic electrons in DNA that produce the experience of conscious awareness and sliding unidirectionally through time.

Decoherence is one of the key novel discoveries of quantum mechanics of recent decades (Ball 2018). This is a practically important concept that allows modeling the biological processes in mesoscale—the scale of macromolecules that have sizes in the shadow zone between the quantum and macroscopic worlds. When purines transform into their aromatic tautomeric forms, their pi electrons are united into an aromatic ring and delocalize. This results in the quantum entanglement of these electrons and increases the coherence of their union. The loss of aromaticity could be caused by the Brownian motion of the nucleoplasm (DNA is constantly bumped by water and other molecules) and by infrared light which is generated by these molecules and fills our bodies. The loss of aromaticity is accompanied by localization (or de-delocalization) of electrons of the aromatic ring, decoherence and collapse of Schrödinger's wave function. Thus purines oscillate between the quantum and macrostates. The quantum delocalized coherent state occurs spontaneously whenever the electrons are left to themselves, which is possible because purines are protected from the outer nucleoplasm by the highly charged backbone of DNA. The macroscopic localized decoherent state is created when Brownian motion or infrared irradiation causes double proton transfer which pulls out an electron from the aromatic ring and causes the ring to fall apart. This way oscillations of aromaticity in DNA provide an interface between the quantum world and the macroscopic world. The DNA can be considered a natural quantum computer and possibly receiver and transmitter of nonlocal quantum information.

Studies of Radin, Sheldrake and others demonstrate that consciousness has a nonlocal component (Sheldrake 2009; Radin 2009; Storm et al. 2017; Bem et al. 2015; Mossbridge and Radin 2018). These studies suggest that not only the brain is involved in the work of the mind. Sheldrake convincingly argues that in addition to the brain, the rest of the body is involved in the work of the mind. For example, there are documented cases in which organ transplants transferred memories and character traits of transplant donors to recipients (Sheldrake 2009; Pearsall, Schwartz, and Russek 2002; Joshi 2011; Liester 2020). Sheldrake also proposed that a substantial part of the human consciousness is located outside of the body in a non-local “morphic field” (Sheldrake 2009). It is suggested herein that coordinated oscillations of aromaticity in stretches of DNA serve as an interface between the local macroscopic and nonlocal “morphic field”.

It has been previously proposed that the genome works as a quantum computer (Richard A. Miller and Webb 1973; Gariaev et al. 2001; Pitkanen 2010). Here, the inventors of the present invention expanded this by adding a specific mechanism for quantum computation. The aromaticity oscillations are coordinated in stretches of DNA and are linked with oscillations of delocalized protons. Both electron and proton clouds are oscillating and they are charged, so these must result in electromagnetic attraction and repulsion which affect the supercoiling of DNA and thus packing of DNA into nucleosomes. In short, aromaticity oscillations control the packing and unpacking of chromatin.

The inventors of the present invention noticed sequence-dependent stacking of aromatic rings and delocalized protons (proton wires) stretched along the base stack and their interactions. It is suggested here that tautomerization would be sequence-dependent and could serve as one of the sequence-dependent oscillators. Moreover, it was noticed that Purines would oscillate between aromatic and nonaromatic states. These effects are utilized in some embodiments of the present invention together with fiberoptic signal transmission, among other things in relation to the work of the mind.

Some embodiments of the present invention include either synthesizing a DNA or modifying a genetic sequence of an organism to contain a desired sequence in large numbers of copies.

According to some embodiments of the present invention it encompasses a system and/or a method wherein aromaticity and ability to bind DNA of a molecule are utilized as a predictor of its potency as a drug. The molecule may intercalate into the DNA. The molecules may be evaluated for the use as drugs for psychological problems, psychiatric problems, cancer, neurological problems, sleep control, temperature control, respiration control, perspiration control, learning ability improvement, addiction, mental performance, emotional control, neuroplasticity, metabolic problems, and fertility. The evaluation can be done computationally or experimentally. The ability of the molecule to change the aromaticity of DNA fragments may be evaluated. Testing of aromaticity of the molecules may be done experimentally using spectroscopy, gel electrophoresis, NMR, infrared spectroscopy, Fourier transform spectroscopy, two-dimensional Fourier-transform infrared spectroscopy, measurements of charge transfer in DNA, measurement of electrochemical properties of DNA in solution or bound to a surface, and impedance spectroscopy of DNA in solution or bound to a surface. The evaluation of the ability of a molecule to change DNA aromaticity may be carried out by computational modeling.

In some embodiments of the invention, it includes a system and/or a method wherein the DNA is used as a processor of a computer which may be a quantum computer, which may be a topological quantum computer. The tautomerization of purines may be utilized for performing logical operations. The quantum computer may be a spintronic computer where the spins of aromatic electrons in purines are utilized for computing. The sequence of DNA may be designed such to position longitudinal hydrogen bonds in DNA to connect two or more basepairs with proton wires and thus create logical circuits. The position purines may be in patterned sequences, such as single nucleotide or multiple nucleotide tandems. The purines patterned sequences may enable coordinated oscillations of aromaticity in multiple basepairs. The logical operations may cause a change in the topology and supercoiling of the double helix.

Some embodiments of the invention include a method and/or a system where histones are added for compacting DNA into nucleosomes. According to some embodiments a DNA sequence-based processor of the quantum computer is incorporated into a living cell such as of a plant, fungi, animal cell culture, yeast, bacteria, single and multicellular organisms, Archaea, Protozoa and Chromista types of cells and organisms. Some embodiments include sending the information into the DNA-based logical circuits and reading the information from the logical circuits for example via optical, infrared, and other wavelengths of electromagnetic and electroacoustic waves and alternating currents. In some cases when the wave patterns are sinusoidal or more complex shapes, where the waves are polarized, optically active (helical) and complex wavefronts are created and analyzed using transmitter and receiver arrays in the solution.

According to some embodiments a so-called mind machine is delivering patterns of light to the eyes of a subject, patterns of sound to the ears of the subject wherein possible, the effects of the mind machine are enhanced by physical therapy applied to various parts of the body. This mind machine may be used for therapy of psychological problems, psychiatric problems, cancer, neurological problems, sleep control, temperature control, respiration control, perspiration control, learning ability improvement, addiction, mental performance, emotional control, neuroplasticity, metabolic problems, and fertility. The mind machine may be used for the improvement of mental performance, for meditation and for entertainment. The patterns of light and sound may be phase-shifted in the relation of the right to left and light to sound. The patterns of light and sound may be controlled by a computer. The patterns of light and sound may be modified based on the electric and bioimpedance measurements from specified locations on the body thus forming a feedback-based treatment. The patterns of light and sound may be dynamically or automatically modified or optimized based on the electric and bioimpedance measurements from specified locations on the skin and body in relation to specified goals or objective functions toward the achievement of certain desired outcomes. The physical therapy in some embodiments of the invention includes LLLT, lightwave patterns, TENS (electric stimulation wave patterns), millimeter-wave patterns, sound, infrasound and ultrasound wave patterns, vibration wave patterns, technological massage movement patterns, infrared waves and heat applied to the skin at specific locations on the body. The mix of such therapies, the individual properties of each (patterns, forms, etc), and relative properties of each (intensities, etc) may dynamically or automatically modified or optimized based on the electric and bioimpedance measurements from specified locations on the skin and body in relation to specified goals or objective functions toward the achievement of certain desired outcomes. The specific locations on the body may be determined using traditional Chinese acupuncture points, neurovascular points and Zakharin-Head zones, and or experimentally mapped on the body for a specific patient using bioimpedance and skin galvanic response measurements. The waves, voltages, vibrations and massage strokes of physical therapy may oscillate in patterns where these patterns are phase-shifted relative to the light and sound wave patterns applied to the eyes and ears of the subject. The mix of such therapies, the individual properties of each (patterns, forms, etc), and relative properties of each (intensities, etc) may be dynamically or automatically modified or optimized based on the electric and bioimpedance measurements from specified locations on the skin and body in relation to specified goals or objective functions toward the achievement of certain desired outcomes.

Some embodiments of the invention include a system and/or a method where sequence-dependent patterns of delocalized protons and delocalized electrons in the base stack of DNA are used to predict functionally important locations in the genome and traits of human individuals based on their genotypes. The function may be a psychological trait, a psychiatric diagnosis, a character trait, mental performance metric, emotional flexibility metric, sleep metric, intelligence quotient, the ability for logical thinking, novelty-seeking trait, intuition metric, social adaptivity metric, ability to follow orders, ability to work in a team, ability for managerial work, ability for science, the ability for research, the ability for repetitive labor, ability to memorize information and recall memories, social abilities, learning abilities, the ability for leadership, speaking ability, writing and reading abilities. Such functional predictions can be made for workers and prospective employees with the intended application to hiring, human resources use, selection of students for educational institutions, selection of partners for marriages, for business relations, for working and sports teams, and assignment of roles in a team, group or organization. The function can be biological, biomolecular, biochemical, physiological, health, wellness, disease-related, cancer, metabolic disorders, neurological disorders, Schizophrenia, Parkinson's, Alsheimer's, Depression, bipolar disorder, attention deficit spectrum, autism spectrum, psychopathy, addictive disorders, PTSD, physical performance, disorders of the blood, lymph, immune, neuroimmune, autoimmune, heart, liver, spleen, adrenal, respiration, vision, hearing, hair loss, dermatological, arthritis, reproductive system disorders, genetic abnormalities, genetic diseases, susceptibility or resistance to infectious diseases, diseases and disorders of the digestive system, dental and orthodontic problems. The functional predictions may be performed with the use of data from genome-wide association scans (GWAS). Genotyped and imputed single nucleotide polymorphisms, deletions, insertions and substitution polymorphisms may be used for functional predictions. In some examples Delocalized Electron Chain Length Metric (DECL metric) is used, where for each individual allele of every genotyped or imputed variation locus, DECL metric is a measure of whether and how much the specific allele shortened or lengthened the uninterrupted stretch of purines, where the Length of the Longest Uninterrupted Stretch (LLUS) of purines immediately adjacent or to the locus of variation (upstream or downstream and in either strand) is determined based on the DNA sequence of the allele. The DECL is calculated as the ratio of the lower of the top two LLUSs to the average of the LLUSs for all alleles in the variation locus. In some examples the Delocalized Proton Chain Length Metric (DPCL metric) is used where for each individual allele of every genotyped or imputed variation locus, DPCL metric is a measure of whether and how much the specific allele shortened or lengthened the Uninterrupted Stretch of Protons (USP), where USP is defined as a stretch of any dinucleotide bonds not interrupted by AT, CG or TA dinucleotide bonds where the Length of the Longest USP (LUSP) is the longest USP immediately adjacent or to the locus of variation (upstream or downstream and in either strand) and is determined based on the DNA sequence of the allele, and where DPCL is calculated as the ratio of the lower of the top two LUSPs to the average of the LUSPs for all alleles in the variation locus. The prediction may be based on the correlation of the functional haplotype of GWAS with DECL metric, DPCL metric and the ratio DECL/DPCL.

Some aspects of the present invention include a system for computationally predicting oscillation patterns of charges in a DNA sequence, including one or more means for computationally predicting proton wires containing longitudinal (coaxial) hydrogen bonds. The proton wires span at least two DNA basepairs. The system includes at least one means for predicting electron wires in the DNA which includes stretches of purines. The system includes one or more means for predicting tautomeric oscillations in the DNA.

According to some embodiments of the above aspect the logical circuits created by the DNA longitudinal hydrogen bonds are connected to a plurality of basepairs with proton wires. The DNA sequence may be designed to include patterns of purines in periodic tandem arrays that improve the efficiency of electromagnetic oscillations in electron wires that are arranged in pattern sequences. The pattern may enable coordinated oscillations of aromaticity in a plurality of basepairs purine stretches. Logical operations may cause a change in topology and supercoiling of the DNA double helix.

According to some aspects of the invention method for computationally predicting oscillation pattern of a DNA sequence, includes predicting proton wires containing longitudinal (coaxial) hydrogen bonds, where the wires spanning at least two DNA basepairs; predicting electron wires in the DNA which include stretches of purines; and predicting tautomeric oscillations in the DNA.

A method exemplifying some embodiments of the invention for medical diagnostics includes designing and producing a DNA sequence that matches the resonance frequencies produced by a specific disorder in the human body; creating a DNA based probe with DNA sequence, with a DNA in solution or in a living cell. The probe, surrounding the DNA with two sensors, e.g., electronic, electromagnetic, or electroaccoustic. The probe is connected to a specific location on a patient's body for example via either electrodes, or a gel, or just physically touching the surface. The DNA sample is allowed to be affected by frequencies coming from the patient. One or more sensors are used for reading the conformational, topological or supercoiling changes in said DNA. Computational analysis of the sensors' output is used for the diagnosis.

What is claimed is:

1. A system for computationally predicting oscillation pattern of charges in a DNA sequence, comprising:
at least one means for computationally predicting proton wires having longitudinal (coaxial) hydrogen bonds in said DNA sequence;
at least one means for predicting electron wires in said DNA sequence, said wires connecting the aromatic rings of DNA basepairs; and
at least one means for predicting tautomeric oscillations in said DNA.

2. The system of claim 1, wherein said DNA sequence is a processor of a quantum computer.

3. The system of claim 1, wherein said quantum computer is topological and spintronic, said computer utilizes spins of purines aromatic electrons.

4. The system of claim 1, wherein tautomerization of said basepairs is utilized for performing logical operations.

5. The system of claim 1, comprising logical circuits created by said DNA longitudinal hydrogen bonds, said hydrogen bonds are connected to a plurality of said basepairs with said proton wires and said electron wires, said DNA sequence having at least one purine stretch patterned in periodic tandem arrays, said pattern enables coordinated oscillations of aromaticity in said purine stretches.

6. The system of claim 4, wherein information input is received in said DNA processor, said processor converts said information input into oscillations selected from the group consisting of electromagnetic, electroacoustic, and combinations thereof.

7. The system of claim 4, wherein output of said logical operations is obtained by at least one electronic module adapted to detect at least one change in conformation, topology and supercoiling of said DNA.

8. The system of claim 2, wherein said processor is incorporated into at least one living cell.

9. The system of claim 1, comprising at least one probe connected to a subject body, wherein said probe comprises:
at least one sensor adapted to measure at least one change in said DNA selected from the group consisting of conformational, topological, supercoiling, and combinations thereof; and
at least one processor adapted to receive and analyze input from said sensor related to said measurement of a change in said DNA,
wherein said DNA sequence matching resonance frequencies caused by at least one subject disorder.

10. A method for computationally predicting oscillation pattern of charges in a DNA sequence, comprising:
computationally predicting proton wires containing longitudinal (coaxial) hydrogen bonds, said wires spanning at least two DNA basepairs;
predicting electron wires in said DNA comprising stretches of purines; and
predicting tautomeric oscillations in said DNA.

11. The method of claim 10, comprising:
incorporating said DNA sequence as a processor in a quantum computer; and
utilizing spins of purines aromatic electrons for computing.

12. The method of claim 11, comprising incorporating said processor in at least one living cell.

13. The method of claim 10, comprising utilizing tautomerization of said basepairs for performing logical operations and utilizing periodic tandem arrays of said purines to improve efficiency of electromagnetic oscillations in said electron wires.

14. The method of claim 10, comprising utilizing waves produced by the said DNA sequence for therapy.

15. The method of claim 14, comprising utilizing said waves for improving mental performance.

16. The method of claim 14, comprising modifying said patterns according to electric and bioimpedance measurements made in parts of a subject body.

17. The method of claim 10, comprising utilizing said waves produced by the said DNA sequence for communicating with at least one brain.
